(19) United States
(12) Patent Application Publication
(10) Pub. No.: US 2003/0020876 A1
(43) Pub. Date: Jan. 30, 2003

(54) Height reconstruction from axial distance data using an ellipse arc-step method

(76) Inventors: Michael J. Smith, Orlando, FL (US); Lance R. Marrou, Orlando, FL (US)

Correspondence Address: MANELLI DENISON & SELTER PLLC, 7th Floor, 2000 M Street, N.W., Washington, DC, 20036-3307, US

(21) Appl. No.: 10/207,118

(22) Filed: Jul. 30, 2002

Related U.S. Application Data
(60) Provisional application No. 60/308,133, filed on Jul. 30, 2001.

(51) Int. Cl. A61B 3/10

(52) U.S. Cl. 351/212

(57) ABSTRACT

A method is provided of reconstructing the height data from the radial distance data gathered by a corneal measurement device such as a corneal topographer or videokeratometer. The present invention performs the height reconstruction using an algorithm that is more accurate than the prior art. The prior art uses the circle arc-step or midpoint height reconstruction algorithms, while the present invention teaches the ellipse arc-step height reconstruction. This is important because it is now known that the eye surface is better modeled with an ellipsoid approximation than a spherical approximation.

This Application is based on and claims priority from U.S. Provisional Application No. 60/308,133 filed on Jul. 30, 2001, the entirety of which is expressly incorporated herein by reference.

### BACKGROUND OF THE INVENTION

1. Field of the Invention

The invention relates to corneal topographers, videokeratometers, and, more particularly, to a method for reconstructing height from axial distance data using an ellipse arc-step method. The present invention also relates to post-processing axial distance data to reconstruct height data after exporting data from a measurement device.

2. Background

Videokeratometers provide the clinician with relevant data on the human eye after an appropriate examination. The relevant data normally includes the axial power and height. The device does not natively measure height. Rather, the height is reconstructed from axial distance and other data.

Prior to the present invention, the state-of-the-art uses a circle arc-step method. Some instances improve this method further by super-sampling the points in a method called the midpoint method. However, this essentially still uses a circular approximation of the eye surface. This is flawed because the eye is rarely spherical (the cross-section of which is a circle). Indeed, the eye is primarily ellipsoidal, as detailed further in the present invention.

### SUMMARY OF THE INVENTION

An object of the invention is to reconstruct height data from axial distance data using an ellipse arc-step method. The axial distance data is measured and used to determine best-fit ellipsoid parameters, which are then used to obtain an accurate approximation of height data for the surface of the eye.

Other objects, features and characteristics of the present invention, as well as the methods of operation and the functions of the related elements of the structure, the combination of parts and economics of manufacture will become more apparent upon consideration of the following detailed description and appended claims with reference to the accompanying drawings, all of which form a part of this specification.

### BRIEF DESCRIPTION OF THE DRAWINGS

The invention will be better understood from the following detailed description of the preferred embodiments thereof, taken in conjunction with the accompanying drawings, in which:

FIG. 1 is a plan view showing an exemplary cross-section of an eye and the relationship between axial distance and height. This figure only illustrates the two dimensional case and shows the X-Z plane.

FIG. 2 is a plan view showing an exemplary cross-section of an eye and an exemplary description of a circle arc-step method, in accordance with the principles of the present invention. FIG. 2 illustrates the two dimensional case and shows the X-Z plane.

FIG. 3 is a flowchart of a method of reconstructing height data from axial distance data using an ellipse arc-step method in accordance with this invention.

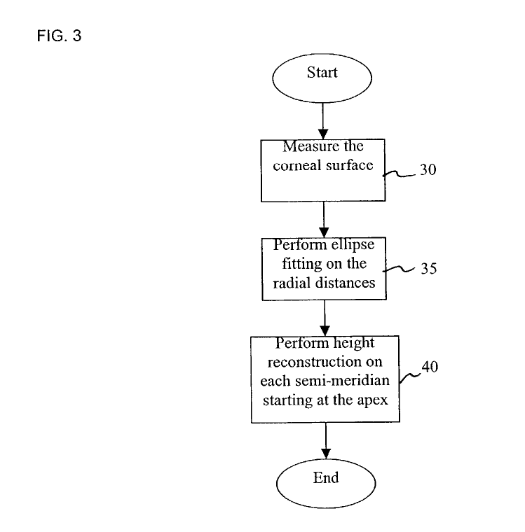

FIG. 4 is an exemplary pseudo-code program illustrating height reconstruction using an ellipse arc-step method in accordance with this invention.

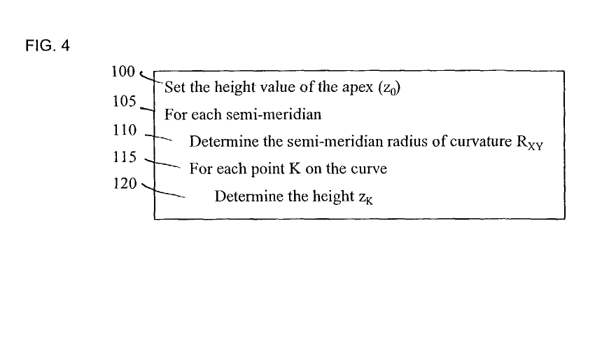

### DETAILED DESCRIPTION OF ILLUSTRATIVE EMBODIMENTS

Two-Dimensional Background

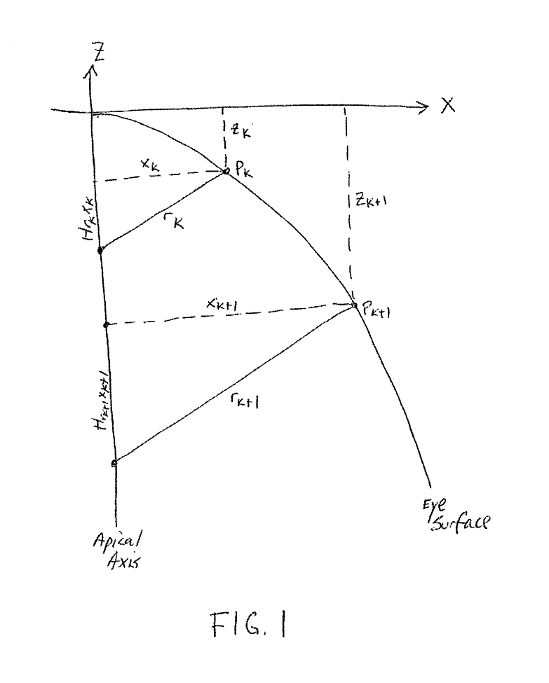

What follows is a detailed description of a method or reconstructing height data in a two-dimensional example. The three-dimensional method is a straightforward extrapolation from this, and is briefly discussed at the end. From FIG. 1, any point P_{K} on the surface of the eye has an associated projected radial distance x_{K}, height to the apex z_{K}, and radial distance r_{K}. The x_{K} and r_{K} values are known. The value for x_{K} is the projected radial distance in the X-Y plane (shown in this exemplary two-dimensional graph along the X-axis). The value for r_{K} is the radial distance measured by the videokeratometer. The value for z_{K} must be computed during height reconstruction.

The value for z₀ is given and is usually set to zero, although the whole surface may be lofted to another known height. Since this is in effect the apical point, the Z-axis is called the apical axis. In practice the apical axis could be the visual axis, optical axis, or any other axis defined in the visual system such that the apex of the surface of the eye would be on that axis. For most videokeratometers, the apical axis is the visual axis because that is the point along which the person sees and is registered with the center point of the device. The arc-step method works by computing the distance between z_{K} and z_{K+}1, accumulating these distances from z₀. The radial distance value is centered along the Z-axis at a height of z_{K}+Hᵣ_{_{K}}ₓ_{_{K}}, as shown in FIG. 1.

To perform height reconstruction, we need to compute the values for Z_{K+1} given the previous value for z_{K}. As stated above, the initial height value z₀ is given, so the first height to find is z₁, or z_{K+1} where K=0. The equation for z_{K+1} depends on the method of approximating the curve between z_{K} and z_{K+1}, however. First, the circle arc-step method is discussed and then the object of this invention, the ellipse arc-step method, is presented. Because the eye more closely resembles an ellipse in most cases rather than a circle, the ellipse arc-step method will be more accurate, thus furthering the state-of-the-art. The midpoint method is more accurate than the normal circle arc-step method, but it still approximates the eye using a circle. The midpoint method can be readily applied to the ellipse arc-step method as well, making it even more accurate.

Circle Arc-Step Method

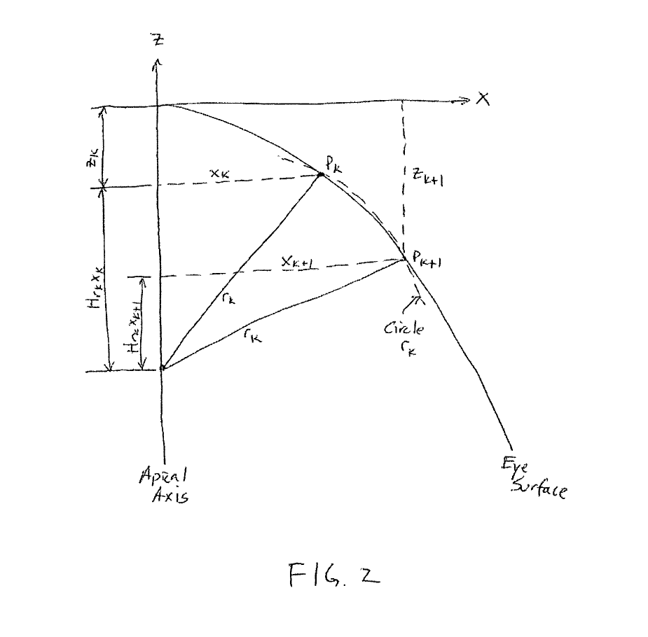

The principle behind the circle arc-step method is that a circle can approximate the curve between two points P_{K} and P_{K+1}. See FIG. 2. An arc of radius r_{K} is shown with its center on the apical axis and passing through the two points P_{K} and P_{K+1}. The center of the arc is at a height of z_{K}+Hᵣ_{_{K}}ₓ_{_{K}}. For each approximation step that the true surface is not well approximated by a circle, error accumulates along the arc. This error is reduced in the midpoint method by halving the arc length at each step. Using the ellipse arc-step method in accordance with the present invention further reduces the error.

Ellipse Arc-Step Method

The ellipse arc-step method uses a similar principle except that the curve between P_{K} and P_{K+1} is approximated by an ellipse. In the two-dimensional case, an ellipse is fitted to the radial distance data directly prior to height reconstruction. This fitting provides us with the ellipse parameters R_{X} and Q. R_{X} is the radius of curvature of the ellipse along the X-axis and the Q is the asphericity parameter, as defined in the equation for an ellipse

$$z = f ( x ) = \sqrt{{( \frac{{R}_{X}}{1 + Q} )}^{2} - \frac{{x}^{2}}{1 + Q}} .$$

The best-fit ellipse can be determined by a number of methods, including solving a linear system of equations or numerical approximation using gradient descent. These methods are applicable to both two-dimensional and three-dimensional algorithms. In order to fit an ellipse to the radial distance, however, the above equation would have to be expressed in terms of radial distance. The value for x is the projected radial distance at each point on the curve.

Expressing the equation for an ellipse in terms of radial distance will be required to get an equation for z in which we can solve for each z_{K+1}. At any point P_{K} on the curve, the radius of curvature R_{K} in terms of the radial distance r_{K}, asphericity Q, and projected radial distance x_{K} is R_{K}=\sqrt{r_{K}²+Qx_{K}²}. This can be substituted for Rₓ in the equation above and simplified as

$$f ( x ) = \frac{1}{1 + Q} \sqrt{{r}_{K}^{2} + Q {x}_{K}^{2} - ( 1 + Q ) {x}^{2}} .$$

Now the height value for any point P_{K+1} can be solved by subtracting (negative Z-axis) the change in z from P_{K} to P_{K+1}. This is expressed as z_{K+1}=z_{K}−(f(x_{K})−f(x_{K+1})). Substituting, we get the equation to solve in the two-dimensional case:

$${z}_{K + 1} = {z}_{K} - \frac{1}{1 + Q} \sqrt{{r}_{K}^{2} - {x}_{K}^{2}} + \frac{1}{1 + Q} \sqrt{{r}_{K}^{2} + Q {x}_{K}^{2} - ( 1 + Q ) {x}_{K + 1}^{2}}$$

Three-Dimensional Extrapolation

The three-dimensional method is a straightforward extrapolation. The value for x_{K} in FIG. 1 is the projected radial distance in the X-Y plane instead of only along the X-axis. Given the X and Y Cartesian coordinates it would be \sqrt{x²+y²}. The surface of the eye is discretized into a number of semi-meridians (preferably one every degree, but it is possibly to create wider subdivisions such as one semi-meridian every two, five or ten degrees). Each semi-meridian creates the cross-section as depicted in FIG. 1. The X-axis becomes the axis along the semi-meridian.

Post-Processing

Ideally, the method outlined in the present invention should be included in the measurement device of the radial distance data, i.e. the videokeratometer. This would provide the most flexibility. However, such an implementation is not always feasible, especially when the manufacturer of the videokeratometer is not performing the height reconstruction. In this instance, the present invention can be applied on a secondary computer system or other device external to the primary measurement device. This would relocate the computation of the height reconstruction.

The foregoing preferred embodiments have been shown and described for the purposes of illustrating the structural and functional principles of the present invention, as well as illustrating the methods of employing the preferred embodiments and are subject to change without departing from such principles. Therefore, this invention includes all modifications encompassed within the spirit of the following claims.

What is claimed is:

1. A method of reconstructing height data from axial distance data, said method comprising:
measuring a corneal surface;
determining a best-fit ellipsoid to said measured corneal surface; and
performing height reconstruction with reference to a single point using an ellipse arc-step method.

2. The method of reconstructing height data from axial distance data according to claim 1, wherein:
said single point is a visual center of said corneal surface.

3. The method of reconstructing height data from axial distance data according to claim 1, wherein said best-fit ellipsoid comprises:
a fit along a visual axis of said corneal surface.

4. The method of reconstructing height data from axial distance data according to claim 1, wherein said best-fit ellipsoid comprises:
a fit along an optical (geometric) axis of said corneal surface.

5. The method of reconstructing height data from axial distance data according to claim 1, wherein said best-fit ellipsoid comprises:
a fit along a pupillary axis of said corneal surface.

6. The method of reconstructing height data from axial distance data according to claim 1, wherein said ellipse arc-step method comprises:
height reconstruction in a three-dimensional domain.

7. The method of reconstructing height data from axial distance data according to claim 1, wherein said ellipse arc-step method comprises:
height reconstruction in a two-dimensional domain.

8. Apparatus for reconstructing height data from axial distance data, comprising:
means for measuring a corneal surface;
means for determining a best-fit ellipsoid to said measured corneal surface; and
means for performing height reconstruction with reference to a single point using an ellipse arc-step method.

9. The apparatus for reconstructing height data from axial distance data according to claim 8, wherein:
said single point is a visual center of said corneal surface.

10. The apparatus for reconstructing height data from axial distance data according to claim 8, wherein said means for determining a best-fit ellipsoid comprises:
means for providing a fit along a visual axis of said corneal surface.

11. The apparatus for reconstructing height data from axial distance data according to claim 8, wherein said means for determining a best-fit ellipsoid comprises:
means for providing a fit along an optical (geometric) axis of said corneal surface.

12. The apparatus for reconstructing height data from axial distance data according to claim 8, wherein said means for determining a best-fit ellipsoid comprises:
means for providing a fit along a pupillary axis of said corneal surface.

13. The apparatus for reconstructing height data from axial distance data according to claim 8, wherein said ellipse arc-step method comprises:
means for providing height reconstruction in a three-dimensional domain.

14. The apparatus for reconstructing height data from axial distance data according to claim 8, wherein said ellipse arc-step method comprises:
means for providing height reconstruction in a two-dimensional domain.
